(19) United States
(12) Patent Application Publication
(10) Pub. No.: US 2007/0213842 A1
(43) Pub. Date: Sep. 13, 2007

(54) PROSTHETIC ARM

(76) Inventor: Randy Scott Simmons, Ontario, CA (US)

Correspondence Address: GRAY ROBINSON, P.A., P.O. Box 2328, FT. LAUDERDALE, FL, 33303-9998, US

(21) Appl. No.: 11/682,493

(22) Filed: Mar. 6, 2007

Related U.S. Application Data
(60) Provisional application No. 60/767,165, filed on Mar. 7, 2006.

(51) Int. Cl. A61F 2/58

(52) U.S. Cl. 623/62; 623/64

(57) ABSTRACT

A prosthetic limb is provided, particularly intended for attachment to the arm of an amputee, comprising a forearm section, a wrist section and a hand section which are structurally and functionally interconnected to simulate a large number of the movements performed by the corresponding natural skeletal structures.

### FIELD OF THE INVENTION

This invention relates to prosthetic arms, and, more particularly, to an arm prosthesis which may be attached below the elbow of an amputee and comprises a forearm section, a wrist section and a hand section collectively having an aesthetically acceptable appearance and being capable of simulating many of the natural movements of the wrist and hands.

### BACKGROUND OF THE INVENTION

Prosthetic devices for the arm, wrist and/or hand have been in use for decades, but designs capable of simulating an appreciable number of the discrete movements of these skeletal structures are still unavailable. Hand prostheses, for example, range from the passive type which simulate the appearance of a natural hand but do not move, to newer myoelectric devices having various combinations of pulleys, cables, linkages and the like with battery-powered operating and control systems. It is common for hand prostheses capable of movement to include a hook or cooperating digits which can grasp an object between them but do little else. In addition to their mechanical limitations, the aesthetic appearance of such devices is unacceptable and creates significant negative psychological and emotional issues for the amputee.

Considering the natural function of the arm below the elbow, for example, a number of discrete motions are performed by the wrist, thumb and fingers. The metacarpalphalangeal joint of the fingers permit flexion and extension, as well as rotation, and the interphalangeal joints allow flexion and extension of the proximal, middle and distal phalanges. Motion of the thumb includes, without limitation, flexion, extension, abduction and abduction at the metacarpalphalangeal joint, as well as flexion and extension of the interphalangeal joint. The wrist is capable of abduction, adduction, flexion, extension and rotation. Prior prosthetic arm designs which include both the wrist and hand, and other prostheses comprising the wrist and hand alone, are capable of simulating only a limited number of the natural motions of these skeletal structures. As a result, the utility of such prostheses is limited.

### SUMMARY OF THE INVENTION

This invention is directed to a prosthetic limb which may be attached to the arm of an amputee below the elbow comprising a forearm section, a wrist section and a hand section which are structurally and functionally interconnected to simulate a large number of the movements performed by the corresponding natural skeletal structures.

In the presently preferred embodiment, the hand section includes a thumb and four fingers coupled to a palm plate. Each finger has a proximal phalanx, a middle phalanx and a distal phalanx connected by joint structures which permit flexion and extension. The thumb includes a joint assembly capable of rotation, adduction/abduction and flexion/extension at a metacarpalphalangeal joint, as well as flexion/extension at an interphalangeal joint.

The wrist section comprises structure permitting pronation/supination, abduction/adduction and flexion/extension of the hand section with respect to the forearm section of the prosthesis. As discussed in detail below, cooperating yokes each connected to a spacer ring form a joint assembly to provide the abduction/adduction and flexion/extension movements. A wrist housing contains structure for rotating the yokes, and, in turn, the hand section.

All of the motions of the wrist section and hand section are controlled by a number of “air muscles” and return springs located in the forearm section of the prosthesis. A source of pressurized air is coupled to the air muscles via manifolds formed with ports connected to solenoid valves. The solenoid valves are operated to supply pressurized air to the air muscles selectively and independently of one another. Each air muscle is connected by a cord or the like to one of the joints in the wrist and hand sections such that when pressurized the air muscle causes the cord to create motion at a respective joint in a desired direction. The return springs are connected to the same joints as the air muscles, and they are effective to move each joint back to its original position upon depressurization of a given air muscle.

The entire prosthesis may be covered by a synthetic material which closely resembles the look and feel of human skin. Cosmetic enhancements may be added such as plastic “fingernails” on the distal phalanges of the fingers and thumb and thread or the like resembling hair on the forearm. The result is a prosthesis which not only simulates a significant number of the natural movements of the lower arm, but is aesthetically acceptable to the amputee.

### BRIEF DESCRIPTION OF THE DRAWINGS

The structure, operation and advantages of the presently preferred embodiment of this invention will become further apparent upon consideration of the following description, taken in conjunction with the accompanying drawings, wherein:

FIG. 1 is perspective view of the prosthetic arm of this invention;

FIG. 2 is a partial, perspective view of the air source, input manifold, exhaust manifold and one of the air muscle-return spring pairs for moving the various joints of the prosthesis;

FIG. 3 is plan view of the input manifold shown in FIG. 2;

FIG. 4 is a view similar to FIG. 2 depicting the air muscle and associated return spring in an expanded position at the top of the Fig., and in a contracted position at the bottom of the Fig.;

FIG. 5 is a side view of one of the fingers of this invention;

FIG. 6 is a view similar to FIG. 6 showing the pivotal motion at the knuckle or metacarpalphalangeal joint;

FIG. 7 is a view similar to FIG. 6 except further depicting the motion at the interphalangeal joints of a finger;

FIG. 7A is an enlarged, front view of one of the interphalangeal joints of the finger of FIG. 7;

FIG. 8 is a perspective view of the thumb of this invention;

FIG. 9 is a side view of the wrist section of the prosthesis herein;

FIG. 10 is a plan view of the wrist section shown in FIG. 9;

FIG. 11 is a cross sectional view of the wrist section herein taken generally along line 11-11 of FIG. 10;

FIG. 12 is a cross sectional view of the wrist section herein taken generally along line 12-12 of FIG. 10;

FIG. 13 is a view similar to FIG. 10 depicting adduction of the wrist section;

FIG. 14 is a view similar to FIG. 13 except depicting abduction of the wrist section;

FIG. 15 is a view similar to FIGS. 9 and 11 illustrating flexion of the wrist section of this invention; and

FIG. 16 is a view similar to FIG. 15 except showing extension of the wrist section.

### DETAILED DESCRIPTION OF THE INVENTION

Referring now to the drawings, the prosthetic arm 10 of this invention comprises a forearm section 12, a wrist section 14 and a hand section 16 which are structurally and functionally interconnected to simulate a large number of the movements performed by the corresponding natural skeletal structures. The discussion below describes each section of the arm 10 separately.

Forearm Section

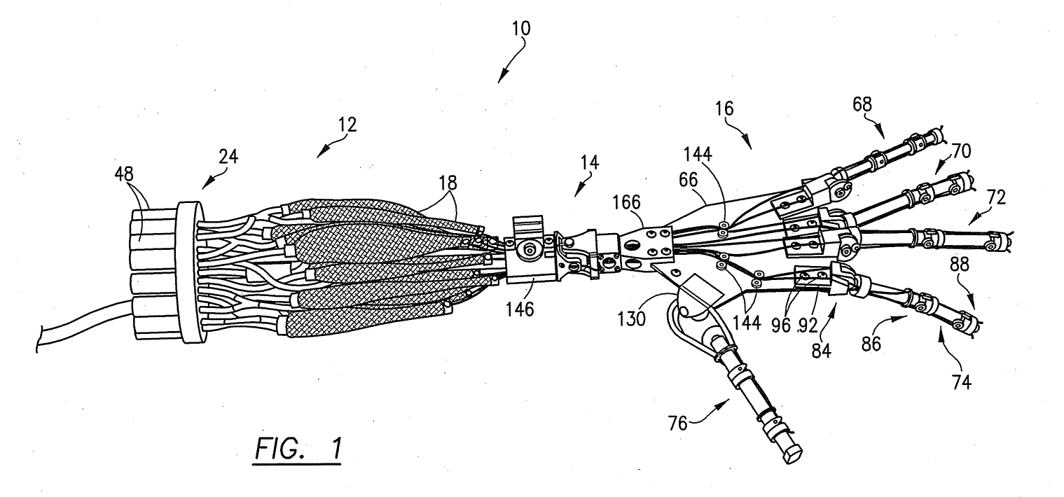

With reference initially to FIGS. 1-4, the forearm section 12 comprises a number of air muscles 18, an equal number of return springs 20, a source of pressurized air 22, an input manifold 24 and an exhaust manifold 26. Considering first the air muscles 18, each is constructed of an elongated sleeve 28 formed of nylon or other flexible material and having a hollow interior 30. A bladder 32 formed of silicon rubber or other material which readily expands and contracts is mounted within the hollow interior 30 of the sleeve 28. The bladder 32 has an open end 34 which is coupled by a fitting 36 to a connector line 38 described in more detail below. The end of the sleeve 28 opposite the fitting 36 is connected to a mount 39 which secures one end of a cord 40 whose opposite end extends to one of the joints of the wrist section 14 or hand section 16 described below. The term “cord” 40 refers to a length of cable, monofilament or essentially any other lightweight and strong material which resists wear.

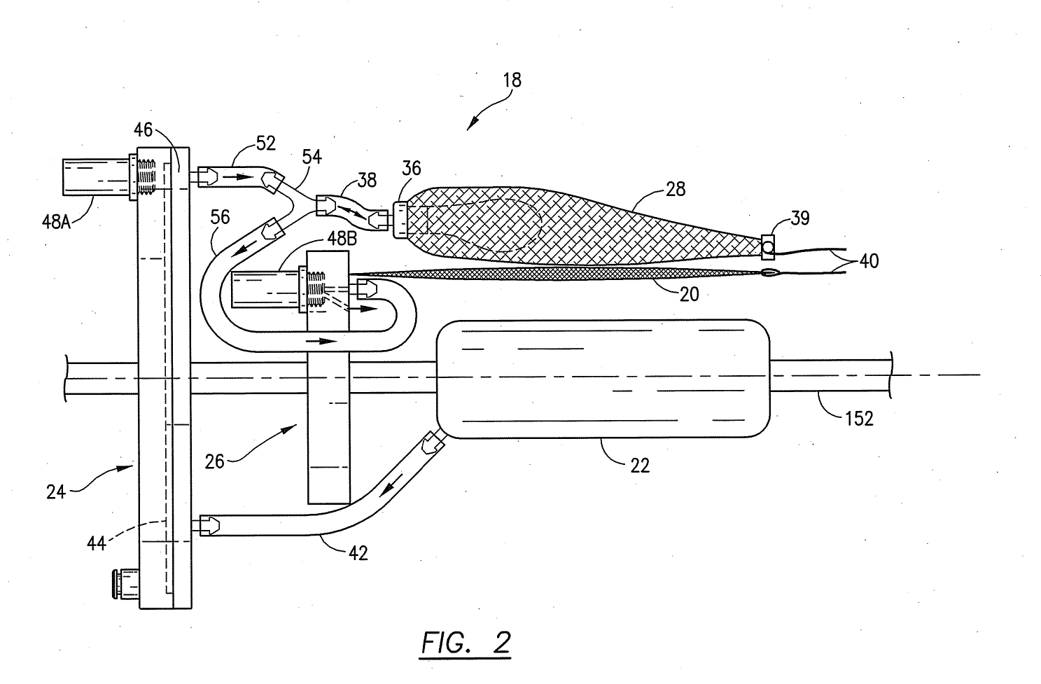

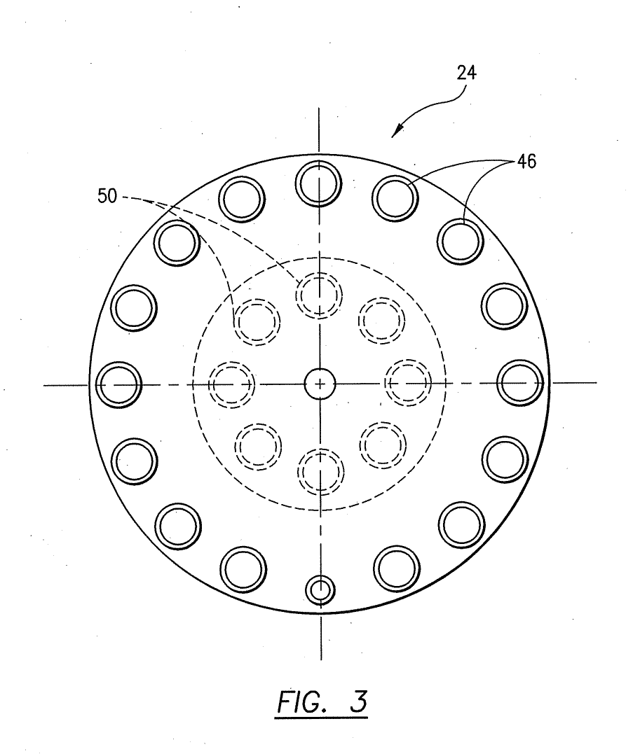

As best seen in FIGS. 2 and 3, the input manifold 24 is connected to the air source 22 by a supply line 42. The air source 22 provides air at a pressure of approximately 20 psi to a circumferentially extending groove 44 formed in the input manifold 24 in communication with a number of spaced inlet ports 46. Consequently, a supply of air at a constant 20 psi is present at each of the inlet ports 46. The inlet ports 46, in turn, each mount a solenoid valve 48 which are normally closed but may be opened in response to a signal. The input manifold 24 may be formed with a number of exhaust ports 50, shown in phantom in FIG. 3, each of which is also connected to a solenoid valve 48. Exhaust ports are also formed in the exhaust manifold 26, each of which mounts a solenoid valve 48, for purposes to become apparent below.

The purpose of the input manifold 24, exhaust manifold 26 and their associated valves 48 is to supply pressurized air to selected air muscles 18. As best seen in FIG. 2, an inlet line 52 extends from one of the inlet ports 46 in the input manifold 24 to one leg of a Y-shaped fitting 54. Another leg of the fitting 54 is coupled to the connector line 38 from the bladder 32, and the third leg of the fitting 54 is coupled to an outlet line 56. The outlet line 56 extends to an exhaust port formed in the exhaust manifold 26. It should be understood that the outlet line 56 may connect to one of the exhaust ports 50 in the input manifold 24 instead of to the exhaust manifold 26. Due to the large number of cords 40 needed to accomplish the joint movements described below, and space limitations relating to the overall size of the forearm section 12, not all of the inlet ports 46 and exhaust ports 50 may be included in a single manifold.

Referring now to FIGS. 3 and 4, in response to a signal opening the valve 48A associated with a selected inlet port 46, pressurized air from the groove 44 in the input manifold is permitted to pass through the inlet port 46 into the inlet line 52. The valve 48B associated with the exhaust port in the exhaust manifold 26 remains in the closed position, thus blocking the flow of air entering the inlet line 52 from escaping through the exhaust manifold 26. The flow of pressurized air is therefore directed through the connector line 38 into the interior of the bladder 32 causing it to become pressurized and move to an expanded position shown at the top of FIG. 4. When expanded, the bladder 32 bears against the wall 58 of the sleeve 28 forcing it to move a distance “L” in the direction of the arrow 60, which, in turn, moves the cord 40 in the same direction. Such motion of the cord 40, as described below, creates a desired movement of a joint in the wrist section 14 or hand section 16 of the arm 10.

The air muscles 18 and return springs 20 operate in pairs. Preferably each return spring 20 comprises a length of elastic material or other material having memory. The return spring 20 depicted in FIG. 4 is connected by a separate cord 40 to the same joint (not shown) as the cord 40 extending from the air muscle 18 in FIG. 4. As seen at the top of such Fig., as the air muscle 18 moves in the direction of arrow 60 the return spring 20 is extended or stretched in the opposite direction, represented by arrow 62. As discussed below, that is because a particular joint has moved from one position to another as a result of pressurization of the air muscle 18, and such displacement of the sleeve 28 induces movement of the return spring 20 in the opposition direction.

After the air muscle 18 depicted in FIG. 4 is pressurized and creates the desired movement of a joint, the valve 48A may be closed thus stopping the flow of air into inlet line 52. At the same time, the valve 48B is opened to provide a flow path for exhausting the air from the bladder 32, i.e. through the connector line 38 and outlet line 56 to an exhaust port in the exhaust manifold 26 where the air can be vented to atmosphere. When the bladder 32 is depressurized, it contracts and moves out of contact with the wall 58 of the sleeve 28. This releases the force applied by the air muscle 18 on the cord 40, and, in turn, on the particular joint being acted upon. The return spring 20, which was previously extended, now exerts a return force on the joint in question via its cord 40 in the direction of arrow 64 causing the sleeve 28 to extend and move in the opposite direction denoted by arrow 66. See bottom of FIG. 4.

As noted above, each joint described below is acted upon by an air muscle 18—return spring 20 pair. The operation of valves 48 on both the input manifold 24 and exhaust manifold 26 is controlled to activate a particular pair of air muscles 18 and return springs 20 so as to obtain the desired movement in the wrist section 14 or hand section 16 of the arm 10. It is contemplated that the arm 10 may be formed with an aperture (not shown) in the area of the forearm section 12 where the air source 22 is located so as to permit refilling of the air source 22 from time to time, as needed.

Hand Section

Referring now to FIGS. 1 and 5-8, the hand section 16 of the prosthetic arm 10 of this invention is shown in detail. The hand section 16 includes a palm plate 66, four fingers 68, 70, 72 and 74, and, a thumb 76. For purposes of the present discussion, only finger 74 is described in detail herein, it being understood that the other fingers 69-72 are structurally and functionally identical. The thumb 76 is described separately below. In each case, the terms used to detail the construction and operation of fingers 68-74 and thumb 76 correspond to the skeletal structure and joints of natural fingers and thumbs. The materials used to construct such elements may be lightweight aluminum, durable plastic or other suitable materials.

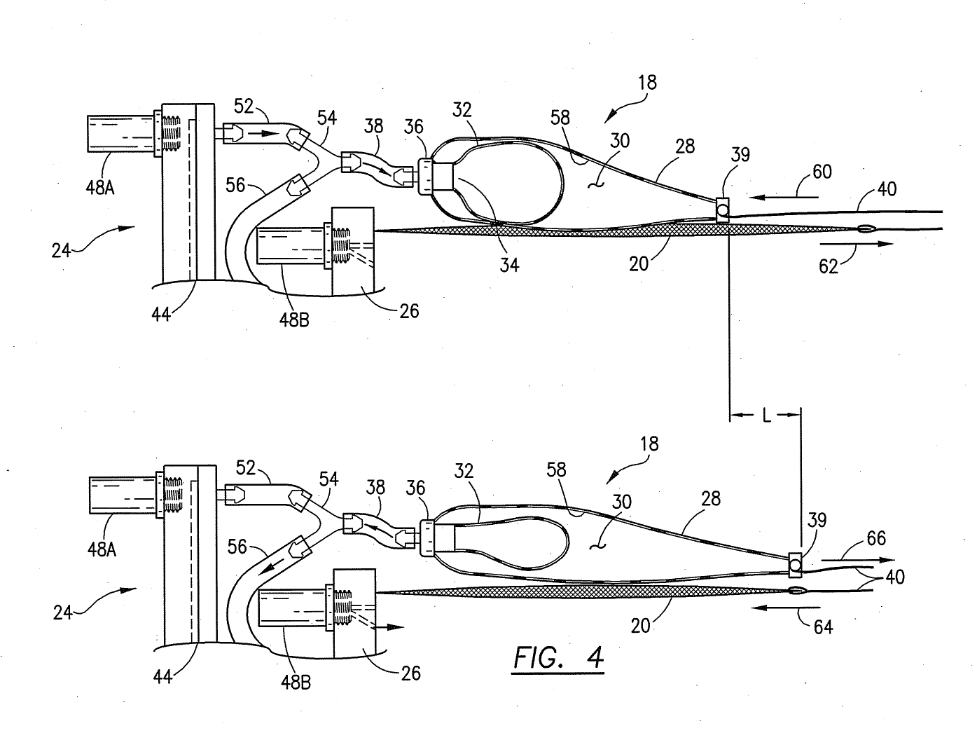

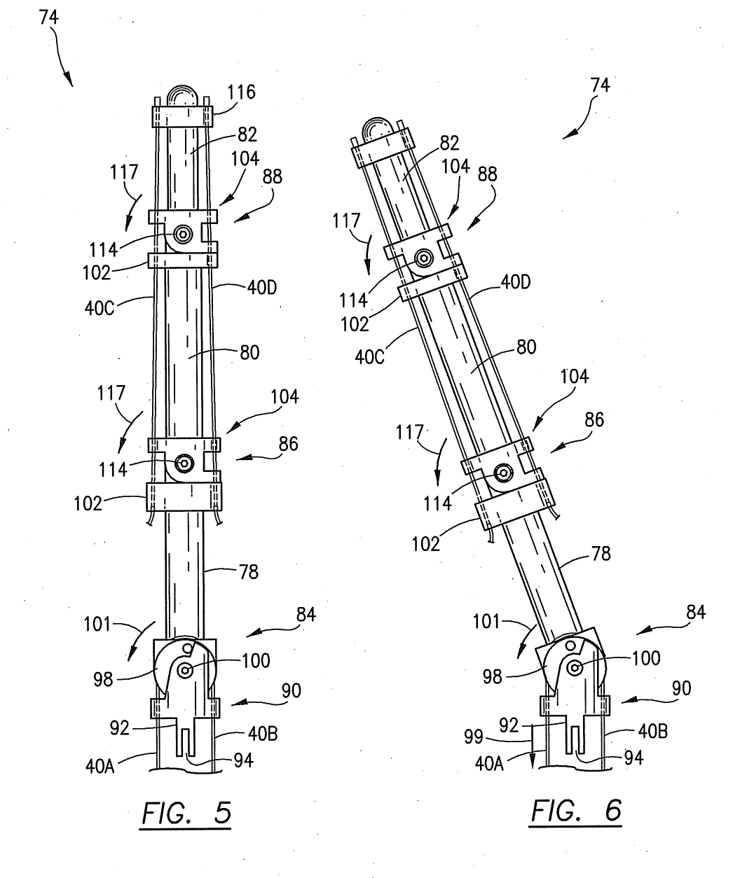

The construction of finger 74 is best seen in FIGS. 5-7. Finger 74 comprises a proximal phalanx 78, a middle phalanx 80 and a distal phalanx 82 each of which may be hollow or solid tubes formed of the materials noted above. A metacarpalphalangeal joint 84 is provided between the proximal phalanx 78 and the palm plate 66, and interphalangeal joints 86 and 88 are provided between the proximal phalanx 78 and middle phalanx 80, and between the middle phalanx 80 and distal phalanx 82, respectively.

Considering first the metacarpalphalangeal joint 84, a mounting block 90 having an end portion 92 formed with a slot 94 is secured to the palm plate 66 with screws 96. The palm plate 66 is received within the slot 94 of the mounting block 90, as depicted in FIG. 1. A pulley 98 is pivotally connected to the mounting block 90 by a bolt or pin 100, and fixed to the proximal end of the proximal phalanx 78. A cord 40A is connected to one side of the pulley 98 which extends from one of the air muscles 18 described below, and a second cord 40B is connected to the opposite side of the pulley 98 extending from one of the return springs 20. In response to operation of the air muscle 18, the cord 40A is pulled in the direction of arrow 99 thus causing the pulley 98 to rotate in a counterclockwise direction as viewed in FIGS. 5 and 6 and as denoted by arrow 101. In turn, the proximal phalanx 78 undergoes flexion, i.e. it pivots in the counterclockwise direction taking with it the middle and distal phalanges 80, 82. See FIG. 6. When the air muscle 18 is depressurized, the return spring 20 rotates the pulley 78 in the clockwise direction thus returning the finger 74 to its extended position shown in FIG. 5.

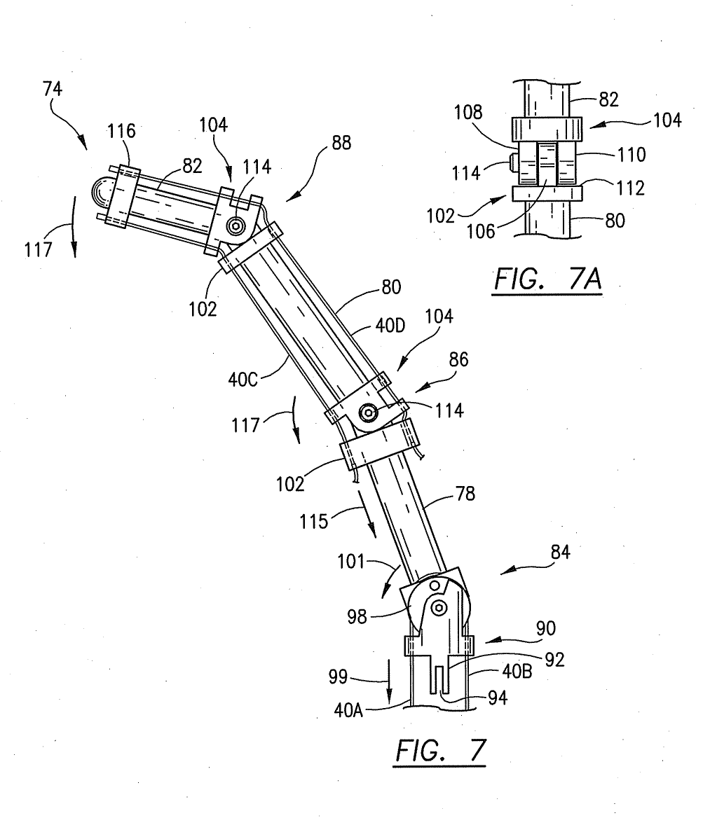

The interphalangeal joints 86 and 88 have the same construction and therefore only one is described herein with the same reference numbers being utilized to denote the same structure in each. The joint 88 comprises a stop 102 fixed at the distal end of the middle phalanx 80, and a pivot element 104 fixed at the proximal end of the distal phalanx 82. As best seen in FIG. 7A, the stop 102 has an extension 106 extending outwardly from the center thereof which is received between opposed arms 108 and 110 formed in the pivot element 104. The distal end of the stop 102 acts as a bearing surface 112 against which the arms 108 and 110 may pivot. This pivotal motion is permitted by a bolt or pin 114 which connects the arms 108 and 110 with the extension 106.

In the presently preferred embodiment, both of the joints 86 and 88 are operated together by cords 40C and 40D. Bores are formed in the stops 102 and the pivot element 104 of each joint 86 and 88 through which the cords 40C and 40D are extended, and then the cords 40C, 40D are secured to a plate 116 mounted at the end of the distal phalanx 82. In response to pressurization of an air muscle 18, the cord 40C is pulled in the direction of arrow 115 toward the forearm section 12 of the arm 10 thus causing the pivot element 104 of both joints 86 and 88 to pivot on the bearing surface 112 of the stop 102 in the direction of arrows 117. The middle phalanx 80 and distal phalanx 82 undergo flexion as a result of such pivotal motion. See FIG. 7. When the air muscle 18 is depressurized, the return spring 20 connected to cord 40D exerts a force causing the pivot elements 104 to pivot in the opposite, clockwise direction thus returning the middle and distal phalanges 80, 82 to a fully extended position depicted in FIG. 5. It should be understood that while only one cable 40C is provided to flex the phalanges 80, 82, and one cable 40D returns them to the extended position, additional cords may be employed so that each of the middle and distal phalanges 80, 82 may be separately actuated.

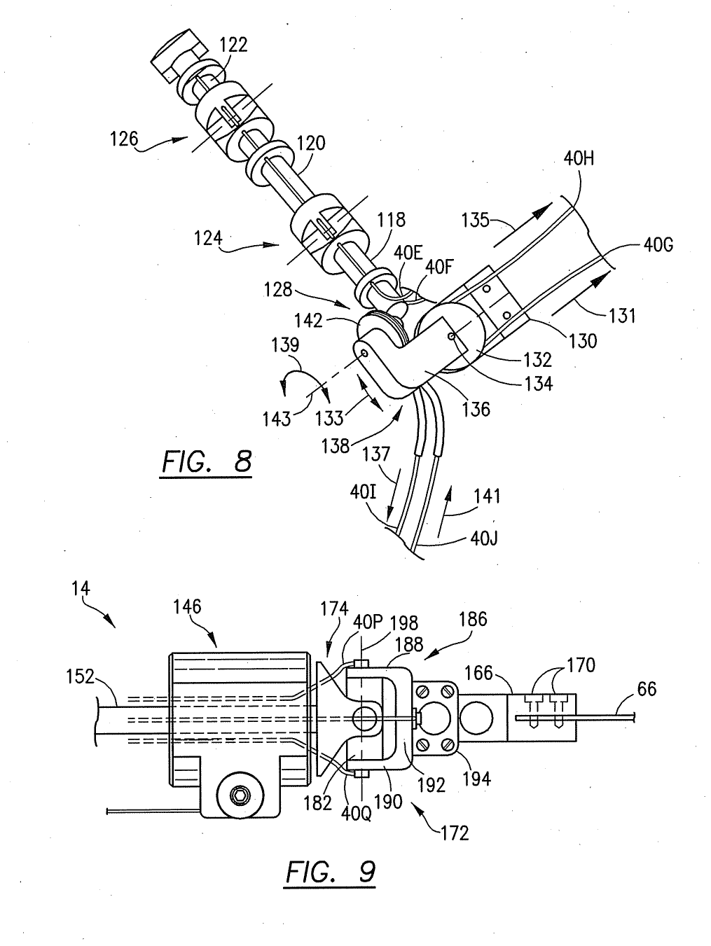

Referring now to FIG. 8, details of the thumb 76 in the hand section 16 of the arm 10 are shown. The thumb 76 includes a metacarpal 118, a proximal phalanx 120, a distal phalanx 122, a metacarpalphalangeal joint 124, an interphalangeal joint 126 and a proximal joint assembly 128. The joints 124 and 126 permit interphalangeal flexion/extension, whereas the joint assembly 128 provides for abduction/adduction and flexion/extension of the thumb 76.

Considering first the joints 124 and 126, the same construction described above in connection with a discussion of the interphalangeal joints 86 and 88 of the finger 74 is employed in joints 124 and 126, and therefore the detailed construction of same is not shown or discussed herein. A cord 40E from an air muscle 18 is affixed to one side of the joints 124, 126, and in response to pressurization of the air muscle 18 the distal phalanx 122 and proximal phalanx 124 undergo flexion, e.g. rotate in a counterclockwise direction in the orientation of thumb 76 depicted in FIG. 8. A return spring 20 connected to cord 40F is effective to flex the thumb 76 upon depressurization of the air muscle 18, thus returning it to the position shown in FIG. 8.

The proximal joint assembly 128 comprises a mounting plate 130 which connects to the palm plate 66 of the hand section 16. A first pulley 132 is pivotally mounted to the plate 130 by a pin 134, and fixed to one leg 136 of an L-shaped pivot block 138. The other leg 140 of the pivot block 138 pivotally mounts a second pulley 142 which is fixed to the proximal end of the metacarpal 118. One pair of cords 40G and 40H is connected to the first pulley 132 to produce abduction and adduction of the thumb 76. In response to pressurization of an air muscle 18, the cord 40G moves in the direction of arrow 131 which rotates the first pulley 132 about the longitudinal axis of the pin 134 in a counterclockwise direction, i.e. as depicted by arrow 137. This causes the thumb 76 to undergo abduction, i.e. to move away from the palm plate 66 in a perpendicular direction. As the first pulley 132 rotates, the pivot block 138 also rotates about pin 134 thus causing the thumb 76 to move in the same direction through its connection to the pivot block 138 via the second pulley 142. When the air muscle 18 is depressurized, the cord 40H connected to a return spring 20 is moved in the direction of arrow 135. In turn, the first pulley 132 is rotated in the clockwise direction causing the thumb 76, through its connection to the pivot block 138, to undergo adduction, i.e. movement to a “neutral” position against the palm plate 66 and in the same plane.

Still another pair of cords 40I and 40J is employed to achieve flexion and extension of the thumb 76. The cord 40I is mounted to one side of the second pulley 142 and the cord 40J connects to its opposite side. Operation of an air muscle 18 pulls on the cord 40I in the direction of arrow 137 causing the second pulley 142 to rotate in a counterclockwise direction about an axis 143 which is perpendicular to the longitudinal axis of pin 134. See arrow 139. This motion flexes the thumb 76, e.g. moves it in a direction across the palm plate 66 in the same plane. When the force on cord 40I is released by the air muscle 18, a return spring 20 acts on the cord 40J in the direction of arrow 141 to rotate the second pulley 142 in the opposite or clockwise direction. This motion extends the thumb 76, i.e. moves in a direction away from the palm plate 66 in the same plane.

The cords 40A-40J described above extend from the forearm section 12 of the arm 10, through the wrist section 14, as described in more detail below, and then along both the upper and lower surfaces of the palm plate 66 of the hand section 16. A number of guide rollers 144 are provided on each surface of the palm plate 66 to direct the cords 40A-40J to respective fingers 68-74 and to the thumb 76.

Wrist Section

Referring now to FIGS. 9-16, the wrist section 14 of this invention is shown in detail. As an overview, the wrist section 14 is capable of moving the hand section 16 so that it undergoes pronation/supination, flexion/extension and abduction/adduction. Additionally, the wrist section 16 includes structure for guiding the cords 40 from the forearm section 12 to the hand section 16 in a compact and efficient manner.

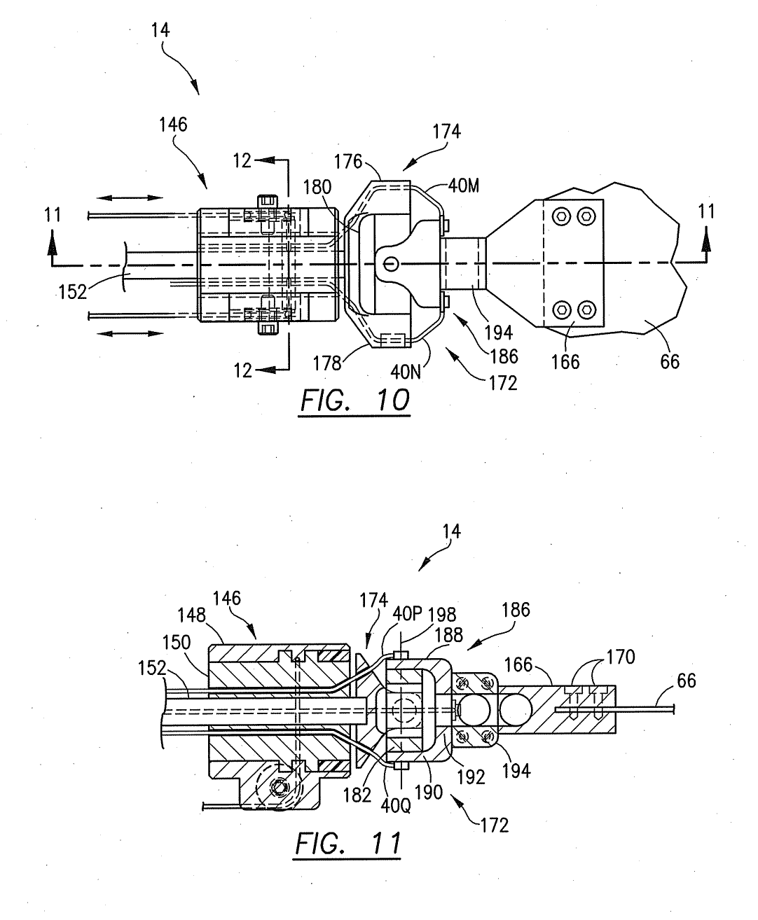

As best seen in FIG. 12, the wrist section 14 includes a wrist housing 146 having an outer casing 148 formed with a hollow interior which receives an inner cord guide 150. The inner cord guide 150 is rotatable with respect to a central rod 152 that extends from the forearm section 12 of the arm 10. Spaced openings 154 and 156 are formed on opposite sides of the inner cord guide 150, each of which receives a number of cables 40 (not shown) connected to the air muscles 18 and return springs 20 as discussed in detail above. The openings 154 and 156 arrange the cords 40 in a compact bundle and direct them to other parts of the wrist section 14, described below, and to the hand section 16.

A first pulley 158 is mounted to one side of the outer casing 148 by a bolt 160, and a second pulley 162 is mounted by a bolt 164 to the opposite side of the outer casing 148. A cord 40K extends around the first pulley 158 and is secured to one side of the inner cord guide 150, while a second cord 40L is trained over the second pulley 162 and connects to opposite side of the inner cord guide 150. Actuation of one of the air muscles 18 pulls on the cord 40K causing the inner cord guide 150 to rotate in a clockwise direction, and when the air muscle 18 is depressurized a return spring 20 acts on cord 40L to rotate the inner cord guide 150 in the opposite direction. The inner cord guide 150 connects to the hand section 16 through other structure of the wrist section 14, described below, so that such rotation created by the cords 40K and 40L results in pronation and supination of the hand section 16.

Referring now to FIGS. 9-11, and to FIG. 1, the wrist section 14 includes a clamping plate 166 having a slot 168 which receives the palm plate 66 of the hand section 16. The clamping plate 166 is mounted to the palm plate 66 by a number of screws 170. A pivot assembly 172 is located between the wrist housing 146 and clamping plate 166. As best seen in FIGS. 10 and 11, and beginning immediately adjacent the wrist housing 146, the pivot assembly 172 includes a first yoke 174 having a first arm 176 and a second arm 178 connected by a plate 180. The plate 180 mounts to the central rod 152 about which the inner cord guide 150 of the wrist housing 146 rotates, as discussed above. A ring 182 having a central through bore is pivotally connected to each arm 176 and 178 of the first yoke 174 to permit rotation about an axis 184 denoted by an “X” in FIG. 10, i.e. the axis 184 extends perpendicular to the sheet on which FIG. 10 is drawn. Preferably, the wall of the ring 182 has an outer surface formed with a number of flats (not shown) so that the wall is hexagonal in shape, for example. These flats provide a surface for mounting of the arms 176, 178 of first yoke 174.

As best seen in FIGS. 9 and 11, a second yoke 186 is also pivotally mounted to the ring 182. The second yoke 186 includes two arms 188 and 190 connected by a plate 192, and the arms 188, 190 each engage one of the flats on the outer surface of the ring 182 but at a spacing of 90° from the locations at which the arms 176 and 178 mount to the ring 182. The second yoke 186 is connected by a spacer 194 to the clamping plate 166.

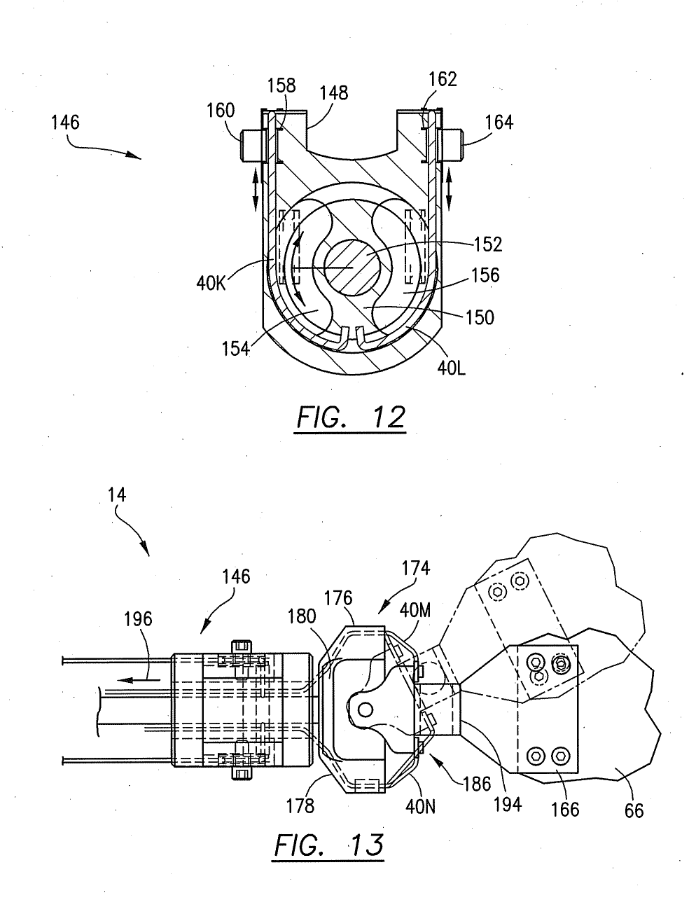

Movement of the second yoke 186 results in abduction/adduction of the hand section 16, or flexion/extension. Referring initially to FIGS. 10, 13 and 14, plan views of the wrist section 14 are provided with a portion of the hand section 16 shown in phantom. For purposes of the present discussion, it is assumed that the hand section 16 is oriented in such Figs. so that the thumb 76 is located at the top of the Figs. and the pinkie finger 68 is located at the bottom. A cord 40M extends from an air muscle 18, through one of the openings 154 or 156 of the inner cord guide 150 to one side of the plate 192 of second yoke 186 where it is secured by a screw or the like. Similarly, a second cord (not shown) connected to a return spring 20 is fed through one of the openings 154 or 156 of the inner cord guide 150 to the opposite side of the plate 192 of second yoke 186 and fastened in place. In response to activation of the air muscle 18, a force is applied to the cord 40M in the direction of arrow 196 in FIG. 13 causing the second yoke 186 to pivot counterclockwise relative to the ring 182 about axis 184. The hand section 16 therefore undergoes adduction, e.g. the movement shown in phantom in FIG. 13. Once the air muscle 18 is depressurized, a return spring 20 pulls on a cord located on the opposite side of the plate 192 so that the hand section 16 returns to a “neutral position” shown in solid lines in FIG. 13. Adduction of the hand section 16, depicted in phantom lines in FIG. 14, is accomplished by actuation of an air muscle 18 to pull on a cord 40N connected to the plate 192 of second yoke 186 opposite to the cord 40M. The second yoke 186 rotates relative to the ring 182 about the axis 184 in a clockwise direction shown in FIG. 14. A return spring 20 connected to a cord (not shown) mounted to the same side of second yoke 186 as cord 40M forces the second yoke 186 back to a neutral position, shown in solid lines in FIG. 14, upon depressurization of the air muscle 18.

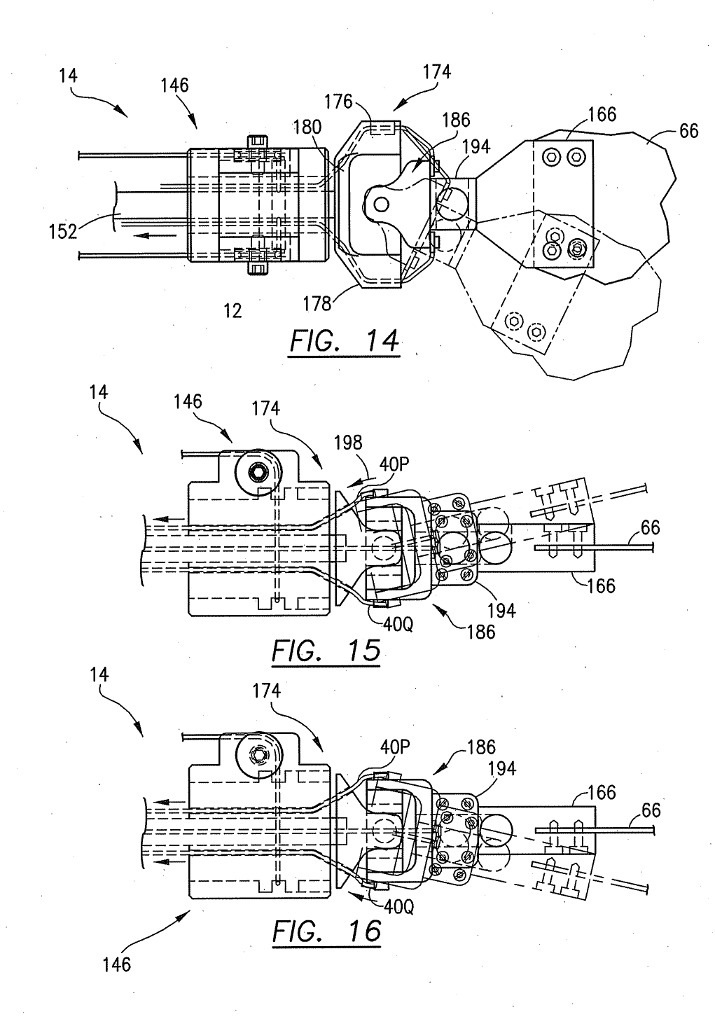

Flexion and extension motions of the hand section 16 created by the wrist section 14 are depicted in FIGS. 11, 15 and 16. A side view of the wrist section 14 is provided in FIG. 11 illustrating the connection of two additional cords, 40P and 40Q, to the arms 188 and 190 of second yoke 186, respectively. Upon pressurization of an air muscle 18 connected to the cord 40P secured to arm 188, the second yoke 186 rotates in the counterclockwise direction depicted by the arrow 198 in FIG. 15. The second yoke 186 is free to rotate about an axis 198, depicted in phantom lines in FIG. 11, because the ring 182 to which it is connected is pivotal with respect to the first yoke 174. Rotation of the second yoke 186 about axis 198 results in flexion of the hand section 16 as illustrated with phantom lines in FIG. 15. When the air muscle 18 is depressurized, return spring 20 connected by a cord (not shown) to the opposite arm 190 of the second yoke 182 returns it to the neutral position depicted by solid lines in FIG. 15. Extension of the hand section 16 occurs in just the opposite manner. The cord 40Q secured to the arm 190 of second yoke 186 is subjected to a force by activation of an air muscle 18 causing pivotal motion of the second yoke 186 and hand section 16 in the clockwise direction seen in phantom lines in FIG. 16. The second yoke 186 and hand section 16 are returned to the neutral position, shown in solid lines in FIG. 16, by operation of a return spring 20 on another cord (not shown) connected to the opposite arm 188 of second yoke 186.

While the invention has been described with reference to a preferred embodiment, it should be understood by those skilled in the art that various changes may be made and equivalents substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A prosthetic arm, comprising:
a forearm section adapted to mount to the arm of a patient;
a hand section including a thumb and at least one finger, said thumb having at least one thumb joint to permit motion of said thumb, said at least one finger having at least one finger joint to permit motion of said at least one finger;
a wrist section connected between said forearm section and said hand section, said wrist section having at least one wrist joint to permit motion of said hand section relative to said forearm section;
a source of pressurized air;
a number of air muscles coupled to said source of pressurized air, each of said air muscles being connected by a cord to one of said at least one finger joint, thumb joint and wrist joint, said air muscles being individually and selectively movable between an expanded position when pressurized wherein said cord associated with an air muscle exerts a force on a respective finger joint, thumb joint or wrist joint, and, a contracted position when depressurized wherein the force exerted by said cord is released; and
a number of return springs each connected by a cord to one of said at least one finger joint, thumb joint and wrist joint, said return springs each being effective to exert a return force on a respective finger joint, thumb joint or wrist joint upon movement of said air muscles to said contracted position.

2. The prosthetic arm of claim 1 in which each of said air muscles comprises a sleeve having a wall defining a hollow interior, and a bladder located within said hollow interior of said sleeve.

3. The prosthetic arm of claim 2 in which said bladder is coupled to said source of pressurized air, said bladder being movable to an expanded position against said wall of said sleeve to cause a cord connected to said sleeve to move in a first direction, said bladder being movable to a contracted position in which said bladder is spaced from said wall of said sleeve.

4. The prosthetic arm of claim 3 further including an inlet manifold coupled to said source of pressurized air and an outlet manifold, each of said inlet and outlet manifolds being formed with a number of ports, at least some of said ports in said inlet manifold each being coupled to a first valve, and said ports in said outlet manifold each being coupled to a second valve, said bladder of each of said air muscles being coupled to one of said ports of said inlet manifold to receive pressurized air from said source, said bladder of each of said air muscles being coupled to one of said ports of said outlet manifold to exhaust pressurized air from said bladder.

5. The prosthetic arm of claim 4 in which said inlet manifold includes at least some ports for exhausting air from said bladder of an air muscle.

6. The prosthetic arm of claim 4 in which said valves coupled to said ports of said inlet and outlet manifolds are solenoid valves.

7. The prosthetic arm of claim 4 further including a fitting, said fitting being connected by a first line to a port of said inlet manifold, by a second line to said bladder of one of said air muscles, and by a third line to a port of said outlet manifold.

8. The prosthetic arm of claim 7 in which said first valve associated with said port connected to said first line opens while said second valve associated with said port connected to said third line closes to permit the flow of pressurized air into said bladder, said first valve closing while said second valve opens to permit the exhaust of pressurized air from said bladder.

9. The prosthetic arm of claim 1 in which said air muscles and said return springs are located in said forearm section.

10. The prosthetic arm of claim 1 in which each of said return springs is a length of elastic material which is extended in response to movement of an air muscle to said expanded position, said elastic material returning to its original length upon movement of said air muscle to said contracted position.

11. The prosthetic arm of claim 1 in which said at least one finger comprises four fingers each having a proximal phalanx, a middle phalanx and distal phalanx, a proximal finger joint being provided between said proximal and middle phalanges of each finger which includes a first stop fixed to said proximal phalanx and a first pivot element connected to said middle phalanx and being pivotal relative to said first stop, a distal finger joint being provided between said middle and distal phalanges which includes a second stop fixed to said middle phalanx and a second pivot element connected to said distal phalanx and being pivotal relative to said second stop.

12. The prosthetic arm of claim 11 in which each of said first and second stops includes an extension extending outwardly from a bearing surface thereof.

13. The prosthetic arm of claim 12 in which each of said first and second pivot elements includes a pair of plates spaced to receive said extension of one of said first and second stops.

14. The prosthetic arm of claim 11 in which said hand section further includes a palm plate, each of said fingers having a metacarpalphalangeal joint located between said palm plate and said proximal phalanx.

15. The prosthetic arm of claim 12 in which each of said metacarpalphalangeal joints includes a mounting plate fixed to said palm plate and a pivot block connected to said proximal phalanx and being pivotal relative to said knuckle mounting block.

16. A prosthetic arm comprising:
a forearm section adapted to mount to the arm of a patient;
a hand section including a thumb and at least one finger, said thumb having at least one thumb joint to permit motion of said thumb, said at least one finger having at least one finger joint to permit motion of said at least one finger;
a wrist section connected between said forearm section and said hand section, said wrist section having at least one wrist joint to permit motion of said hand section relative to said forearm section;
a source of pressurized air;
a number of air muscles each comprising:
(i) a flexible sleeve having a wall defining a hollow interior, said sleeve being connected by a cord to one of said at least one finger joint, thumb joint and wrist joint;
(ii) a bladder located within said hollow interior of said sleeve and being coupled to said source of pressurized air;
(iii) said bladder of each of said air muscles being movable to an expanded position when pressurized and into contact with said wall of said sleeve causing said cord connected to said sleeve to move; and
(iv) said bladder of each of said air muscles being movable to a contracted position out of engagement with said wall of said sleeve when depressurized.
a number of return springs each connected by a cord to one of said at least one finger joint, thumb joint and wrist joint, said return springs each being effective to exert a return force on a respective finger joint, thumb joint or wrist joint upon movement of said bladder of a respective one of said air muscles to said contracted position.

17. The prosthetic arm of claim 16 further including an inlet manifold coupled to said source of pressurized air and an outlet manifold, each of said inlet and outlet manifolds being formed with a number of ports, at least some of said ports in said inlet manifold each being coupled to a first valve, and said ports in said outlet manifold each being coupled to a second valve, said bladder of each of said air muscles being coupled to one of said ports of said inlet manifold to receive pressurized air from said source, said bladder of each of said air muscles being coupled to one of said ports of said outlet manifold to exhaust pressurized air from said bladder.

18. The prosthetic arm of claim 17 in which said inlet manifold includes at least some ports for exhausting air from said bladder of an air muscle.

19. The prosthetic arm of claim 16 in which each of said return springs is a length of elastic material which is extended in response to movement of an air muscle to said expanded position, said bladder returning to its original length upon movement of said air muscle to said contracted position

20. A prosthetic arm, comprising:
a forearm section adapted to mount to the arm of a patient;
a hand section including a palm plate, a thumb and at least one finger, said at least one finger having at least one finger joint to permit motion of said at least one finger, said thumb comprising:
(i) a metacarpal, a proximal phalanx and a distal phalanx pivotally connected by interphalangeal joints;
(ii) a pivot assembly connected to said palm plate and to said metacarpal, said pivot assembly being effective to move said metacarpal, said proximal phalanx and said distal phalanx in flexion, extension, abduction and adduction;
a wrist section connected between said forearm section and said hand section, said wrist section having at least one wrist joint to permit motion of said hand section relative to said forearm section;
a source of pressurized air;
a number of air muscles coupled to said source of pressurized air, each of said air muscles being connected by at least one cord to one of said at least one finger joint, to said at least one wrist joint, to one of said interphalangeal joints of said thumb or to said pivot assembly of said thumb, said air muscles being individually and selectively movable to an expanded position when pressurized wherein said cord associated with an air muscle exerts a force on a respective joint, and to a contracted position when depressurized wherein the force exerted by said cord is released;
a number of return springs each connected by a cord to one of said at least one finger joint, to said at least one wrist joint, to one of said interphalangeal joints of said thumb or to said pivot assembly of said thumb, said return springs each being effective to exert a return force on a respective joint upon movement of said air muscles to said contracted position.

21. The prosthetic arm of claim 20 in which said metacarpal and said proximal phalanx are pivotally connected by a first phalangeal joint, and said proximal phalanx and said distal phalanx are pivotally connected by a second phalangeal joint.

22. The prosthetic arm of claim 21 in which said first and second phalangeal joints permit flexion and extension of said proximal and distal phalanges.

23. The prosthetic arm of claim 20 in which said pivot assembly comprises:
(i) a first pulley connected to said palm plate and rotatable relative to a first axis;
(ii) a second pulley connected to said metacarpal and rotatable relative to a second axis which is perpendicular to said first axis;
(iii) a pivot block having a first leg connected to a second leg, said first leg being fixed to said first pulley and rotatable therewith, said second leg being connected to said second pulley so that said second pulley is rotatable with respect to said second leg.

24. The prosthetic arm of claim 23 in which said first pulley is connected to a cord extending from one of said air muscles, same first pulley causing said pivot block and, in turn, said metacarpal, proximal phalanx and distal phalanx to undergo adduction or abduction in response to movement of said air muscle to said expanded position.

25. The prosthetic arm of claim 23 in which said second pulley is connected to a cord extending from one of said air muscles, same second pulley causing said pivot block and, in turn, said metacarpal, proximal phalanx and distal phalanx, to undergo flexion or extension in response to movement of said air muscle to said expanded position.

26. The prosthetic arm of claim 20 in which each of said interphalangeal joints of said thumb comprises a stop fixed to one of said metacarpal and said proximal phalanx, and a pivot element connected to one of said distal and proximal phalanges which is pivotal relative to said stop.

27. The prosthetic arm of claim 20 in which each of said air muscles comprises a sleeve having a wall defining a hollow interior, and a bladder located within said hollow interior of said sleeve.

28. The prosthetic arm of claim 27 in which said bladder is coupled to said source of pressurized air, said bladder being movable to an expanded position against said wall of said sleeve to cause a cord connected to said sleeve to move in a first direction, said bladder being movable to a contracted position in which said bladder is spaced from said wall of said sleeve.

29. A prosthetic arm, comprising:
a forearm section adapted to mount to the arm of a patient;
a hand section including a palm plate, a thumb and at least one finger, said thumb having at least one thumb joint to permit motion of said thumb, said at least one finger having at least one finger joint to permit motion of said at least one finger;
a wrist section connected between said forearm section and said hand section, said wrist section including:
(i) a wrist housing;
(ii) a first yoke connected to said wrist housing;
(iii) a spacer ring mounted to said first yoke and pivotal relative to a first axis;
(iv) a second yoke fixed to said palm plate and pivotally mounted to said spacer ring for movement relative to a second axis which is perpendicular to said first axis;
a source of pressurized air;
a number of air muscles coupled to said source of pressurized air, each of said air muscles being connected by at least one cord to one of said at least one finger joint, to said thumb joint, to a first portion of said second yoke or to a second portion of said second yoke, said air muscles being individually and selectively movable to an expanded position when pressurized wherein an air muscle exerts a force on a respective finger joint, thumb joint, first portion of said second yoke or second portion of said second yoke, and to a contracted position when depressurized wherein the force exerted by said cord is released;
a number of return springs each connected by a cord to one of said at least one finger joint, to said thumb joint, to a third portion of said second yoke or to a fourth portion of said second yoke, said return springs each being effective to exert a return force on a respective at least one finger joint, said thumb joint or said third or fourth portions of said second yoke upon movement of said air muscles to said contracted position.

30. The prosthetic arm of claim 29 in which said wrist housing includes an outer casing formed with a central throughbore, and an inner cord guide located with said central throughbore and rotatable relative to a central rod.

31. The prosthetic arm of claim 30 in which said outer casing mounts a first pulley and a second pulley, said first pulley receiving a cord which mounts to a first portion of said inner cord guide and said second pulley receiving a cord which mounts to a second portion of said inner cord guide, whereby movement of said first cord rotates said inner cord guide in one direction and movement of said second cord rotates said inner cord guide in an opposite direction.

32. The prosthetic arm of claim 30 in which said first yoke is connected to said inner cord guide and rotatable therewith.

33. The prosthetic arm of claim 29 in which said spacer ring has a wall formed with a central bore, said wall having an outer surface.

34. The prosthetic arm of claim 33 in which said first yoke has a first arm and a second arm connected by a plate, each of said first and second arms being connected by a pin to said outer surface of said wall of said spacer ring so that said spacer ring is rotatable about said pins with respect to said first yoke.

35. The prosthetic arm of claim 34 in which said second yoke has a first arm and a second arm connected by a plate, each of said first and second arms being connected by a pin to said outer surface of said wall of said spacer ring at approximately 90° from the points at which said first and second arms of said first yoke are mounted, said second yoke being rotatable about said pins with respect to said spacer ring.

36. The prosthetic arm of claim 35 in which said first portion of said second yoke comprises one side of said plate which connects said first and second arms of said second yoke, and said third portion of said second yoke comprises the opposite side of said plate which connects said first and second arms of said second yoke, whereby movement of said cord connected to said one side of said plate causes adduction of said palm plate and movement of said cord connected to said opposite side of said plate cause abduction of said palm plate.

37. The prosthetic arm of claim 35 in which said second portion of said second yoke comprises said first arm thereof and said fourth portion of said second yoke comprises said second arm thereof, whereby movement of said cord connected to said first arm of said second yoke causes flexion of said palm plate and movement of said cord connected to said second arm of said second yoke causes extension of said palm plate.
